(19) United States
(12) Patent Application Publication
(10) Pub. No.: US 2009/0311226 A1
(43) Pub. Date: Dec. 17, 2009

(54) COMPOSITION AND METHOD FOR TREATING ESOPHAGEAL DYSPLASIA

(75) Inventors: John W. Harmon, Baltimore, MD (US); Guy Pierre Marti, Cockeysville, MD (US); Tomoharu Miyashita, Baltimore, MD (US); Michael Gibson, Baltimore, MD (US); Todd Armstrong, Elkridge, MD (US); Pramod Bonde, Abingdon, MD (US)

Correspondence Address: BANNER & WITCOFF, LTD., 1100 13th STREET, N.W., SUITE 1200, WASHINGTON, DC, 20005-4051, US

(73) Assignee: THE JOHN HOPKINS UNIVERSITY, BALTIMORE, MD (US)

(21) Appl. No.: 12/300,685

(22) PCT Filed: May 22, 2007
(86) PCT No.: PCT/US07/12137

§ 371 Date: Apr. 24, 2009

Related U.S. Application Data
(60) Provisional application No. 60/802,351, filed on May 22, 2006.

(51) Int. Cl. A61K 48/00; A61P 35/00; A61K 35/00; A61K 35/12

(52) U.S. Cl. 424/93.21

(57) ABSTRACT

Enhancing the immune response to esophageal tumor cells reduces the incidence of esophageal cancer developing, recurring, or metastasizing. Esophageal cancer cells are modified to render them more immunogenic and proliferation compromised. They are used in an antigenic preparation to raise a T cell response in the recipient.

This application claims the benefit of U.S. provisional application Ser. No. 60/802,351 filed May 22, 2006, the disclosure of which is expressly incorporated herein.

### TECHNICAL FIELD OF THE INVENTION

This invention is related to the area of esophageal disease. In particular, it relates to treatment and prevention of development of esophageal metaplasia and cancer.

### BACKGROUND OF THE INVENTION

Esophageal cancer, especially adenocarcinoma, is a disease with increasing incidence and high mortality in the Western hemisphere¹⁻³. At diagnosis, approximately 50% of patients have advanced, incurable disease, while of those with locally advanced disease, at most 30%, are cured with modern combined modality therapy⁴. With the development of molecular medicine and tumor immunology, targeted therapies with small molecules and immunologic approaches with monoclonal antibodies, and tumor vaccines are under investigation for treatment of pre-malignant and malignant neoplasms⁵.

While Barrett's esophagus is a known precursor to esophageal cancer, there is little that can be done to prevent individuals with Barrett's esophagus from progressing to cancer. For low grade dysplastic Barrett's, as for Barrett's without dysplasia, surveillance is the standard management. With high grade dysplasia, esophagectomy is often recommended^{18,19}. Other ablative modalities such as those using photodynamic, endoscopic, or laser treatments are in clinical evaluation²⁰. Clearly if a tumor vaccine could be shown to prevent progression of Barrett's metaplasia to cancer there would be many individuals who could benefit from such preventive treatment.

There is a continuing need in the art to improve the treatment and prevention of esophageal cancer.

### SUMMARY OF THE INVENTION

According to one embodiment of the invention a composition is provided. The composition comprises mammalian esophageal cancer cells which express MHC I molecules and which express GM-CSF from an exogenous expression construct. The cells are made propagation-defective prior to administration to a mammal.

According to another embodiment of the invention a method of treating a mammal with esophageal cancer, with Barrett's metaplasia, or prone to develop Barrett's metaplasia, is provided. A composition is administered to the mammal whereby risk that the mammal develops esophageal cancer, esophageal cancer recurrence, or esophageal cancer metastases is reduced. The composition comprises propagation-defective mammalian esophageal cancer cells which express MHC I molecules and which express GM-CSF from an exogenous expression construct.

These and other embodiments which will be apparent to those of skill in the art upon reading the specification provide the art with methods and therapeutic and prophylactic compositions for treating individuals that may develop primary esophageal cancer, its recurrence, or metastases.

### BRIEF DESCRIPTION OF THE DRAWINGS

FIG. 1: A timeline of the implant experiment. A vaccination or PBS injection was delivered seven days prior to the start of the experiment. Tumors were transplanted in all of the rats on day 0 and were measured on day 0, 4, 7, 11, and 14. The experiment ended on day 14, when the remaining tumor masses were harvested from all of the animals.

FIG. 2: A timeline of the surgical reflux model experiment. A vaccination or PBS injection was delivered 20, 24, 30, and 34 weeks after surgery. The experiment ended 40 weeks following post-surgery, when all animals were sacrificed and any remaining tumor masses were harvested from the animals.

FIG. 3: A representative picture from a reflux-induced tumor in a rat that underwent a jejuno-esophagostomy. The tumor was found near the site of anastomosis.

FIG. 4: Flow cytometry confirmed MHC class I expression in both JA and JB cell lines was greater than the Isotype control.

FIG. 5A-5H: A representative photomicrograph of the vaccination site. The infiltration of T-cells, eosinophils (i), macrophages, and granulomas suggests the GM-CSF secreting vaccine promoted an inflammatory response in rats (FIG. 5A and FIG. 5C). A sham vaccination with the same cancer cells not transfected to express GM-CSF promoted necrosis and a smaller inflammatory response (FIG. 5B and FIG. 5D). Immunohistochemical staining showed that the irradiated GM-CSF secreting vaccine cells promoted a robust infiltration of CD8⁺ T-cells at the vaccination site (FIG. 5 E). There was minimal infiltration of CD8⁺ T-cells in animals belonging to the group injected in the identical manner with irradiated cells that had not been transfected to express GM-CSF (FIG. 5 F). Granuloma size and eosinophil counts in rats belonging to the GM-CSF vaccinated group were significantly larger than those of non-transfected placebo animals (FIG. 5G and FIG. 5H). The proportion of CD8⁺ T cells in five random samples was higher in both the vaccinated groups than in the non-vaccinated GM-CSF (−) placebo control group.

FIG. 6: A representative picture of transplanted tumor sizes in a vaccinated and a non-vaccinated PBS placebo animal throughout the length of the experiment. The tumor grew progressively larger in the non-vaccinated PBS placebo animal over the span of 14 days. In contrast, the tumor in the vaccinated animal disappeared completely by day 4. This rat was re-transplanted with tumor cells on that day. The re-transplanted tumor was promptly destroyed in this animal.

FIG. 7: Tumor volume is graphed as a function of time for non-vaccinated PBS placebo and vaccinated animals. The tumor size on the day 0, the first day of the experiment, represents the injection volume of tumor cells and Matrigel™. Tumors in the non-vaccinated PBS placebo group continued to grow until the end of the experiment, while tumors in four of the six rats in the vaccine group disappeared completely by day 4, and the tumors present on the remaining two animals disappeared by day 14.

FIG. 8: The four rats whose tumors disappeared by day 4 were re-implanted with additional tumor cells on that day. In addition, four naïve animals were implanted for the first time and served as a naïve non-vaccinated PBS placebo group. There was a pronounced decrease in the size of tumors found on vaccinated rats between days 4 and 11 of the experiment. Tumors in naïve non-vaccinated PBS placebo rats increased in size.

FIG. 9A-9B: At day 14, photomicrographs of vaccinated rats displayed an abundance of macrophages and no remaining neoplastic cells (FIG. 9A). On the other hand, a representative photomicrograph displays clear tumor growth in the non-vaccinated PBS placebo group. An abundance of neoplastic cells and keratin pearls (→) can be seen in the non-vaccinated PBS placebo group on day 14 (FIG. 9B).

FIG. 10: On day 11, a vaccinated animal shows macrophages and giant cells being recruited to surround and destroy tumor cells in a rat that was re-transplanted with tumor cells. As a result of the immune response, the tumor cell is undergoing necrosis.

FIG. 11A-11F: Immunohistochemical images show that animals that were irradiated and transfected with GM-CSF expressing cells of tumor transplants had greater infiltration of CD4⁺ T-cells and CD8⁺ T-cells at the tumor transplant sites (FIG. 11A, FIG. 11B) when compared to non-vaccinated PBS placebo animals (FIG. 11C, FIG. 11D). Black arrows (→) indicate distinct tumor nodules. CD4⁺ and CD8⁺ T cells were counted in five random high power fields. Vaccinated animals displayed a greater response when compared to non-vaccinated PBS placebo animals (FIG. 11E, FIG. 11F).

FIG. 12A-12B: Macroscopic findings. (FIG. 12A) The distal portion of an esophagus in a non-vaccinated PBS placebo rat thickened. The epithelium was rough and a transparent tumor is visible near the anastomosis (→). (FIG. 12B) The esophagus of a rat from the vaccine group exhibited a slightly uneven surface, but displayed no tumor.

FIG. 13: Experimental timeline. A vaccination or PBS injection was delivered 4, 6, 14, and 16 weeks after surgery. The experiment ended 40 weeks following post-surgery, when all animals were sacrificed and any remaining tumor masses were harvested from the animals.

FIG. 14A-14B: A representative photomicrograph of an esophageal carcinoma which resulted from surgically induced duodenal contents reflux. FIG. 14A. Proliferative Hyperplasia; FIG. 154. Barrett's metaplasia (→); FIG. 14C. Adenocarcinoma (→); FIG. 14D. Squamous cell carcinoma with keratin pearls (→)

FIG. 15A-15B: Macroscopic findings. (FIG. 15A) The distal portion of an esophagus in a non-vaccinated PBS placebo rat thickened. The epithelium was rough and a transparent tumor is visible near the anastomosis. (FIG. 15B) The esophagus of a rat from the vaccine group exhibited a slightly uneven surface, but displayed no tumor.

### DETAILED DESCRIPTION OF THE INVENTION

The inventors have developed a new method for treating patients who are at risk of developing esophageal cancer, esophageal cancer recurrence, or esophageal cancer metastases. Administration of a composition according to the invention reduces the risk/incidence of developing esophageal cancer, esophageal cancer recurrence, or esophageal cancer metastases. The composition may also prevent the development of Barrett's esophagus.

The composition comprises propagation-defective mammalian esophageal cancer cells. The cancer cells may be derived from a cell line. The cell line may be derived from a primary esophageal tumor or xenograft. The histology of the esophageal cancer from which the cells are derived may be of any type, including but not limited to adenosquamous, adenocarcinoma, and squamous cell. Suitable esophageal cancer cells lines which can be modified and made propagation-defective include without limitation: BIC, SEG, (Aggarwal, Neoplasia. 2000 July-August; 2(4):346-56.) YES-1, (Nippon Geka Hokan. 1991 Jan. 1; 60(1):3-12), KAN-ES (“Establishment of Novel Human Esophageal Cancer Cell Line in Relation to Telomere Dynamics and Telomerase Activity,” Journal Digestive Diseases and Sciences, Volume 45, 2000, Pages 870-879), etc. Any means known in the art for rendering cancer cells propagation-defective may be used, including heat killing, formalin inactivation, gamma irradiation, etc. Any chemical, thermal, or other treatment can be used which preserves antigenic properties of the cancer cells. The propagation-defective property is a safety feature to prevent the inadvertent establishment of live cancer cells from the composition. Assessment of propagation-deficiency can be performed either in culture (in vitro) or in a whole animal (in vivo), such as a xenograft. A stringent level of propagation-deficiency should be maintained for safety purposes.

For irradiation of tumor cells, the tumor cells can be plated in a tissue culture plate and irradiated at room temperature using a ¹³⁷Cs source. The cells can be irradiated at a dose rate of from about 50 to about 200 rads/min, or from about 120 to about 140 rads/min. The cells can be irradiated with a total dose sufficient to impair the replication/propagation of the cells in vitro.

The composition of the present invention comprises esophageal cancer cells which are modified to express GM-CSF from an expression construct. Typically the cells are transfected with a plasmid or viral vector that encodes GM-CSF. Preferably the GM-CSF is of the same species as the subject to be treated. Thus if the composition is to be used to treat humans, a human GM-CSF expression construct will be used; if a rodent is to be treated then a rodent GM-CSF will be used. Other immune stimulatory cytokines can be used without limitation, including but not limited to interferons alpha, beta, or gamma, interleukins, tumor necrosis factors, erythropoietin, M-CSF, and G-CSF. A vector encompasses a DNA molecule such as a plasmid, virus or other vehicle, which contains one or more heterologous or recombinant DNA sequences, e.g., a cytokine gene or cytokine coding sequence of interest under the control of a functional promoter and possibly also an enhancer. Appropriate viral vectors include, but are not limited to simian virus 40, bovine papilloma virus, Epstein-Barr virus, adenovirus, herpes virus, vaccinia virus, Moloney murine leukemia virus, Harvey murine sarcoma virus, murine mammary tumor virus, and Rous sarcoma virus.

Optionally the esophageal cancer cells can be further modified to express a marker protein, such as GFP (green fluorescent protein) or luciferase. Such markers are easily detectable and can facilitate the tracking of the cancer cells in the body of a mammal. Markers may be useful to determine safety of compositions. For example, they may be useful to determine the extent of the treatment rendering cancer cells propagation-defective.

The vector may be stably maintained or relatively stably maintained in the tumor cell. Alternatively, the vector may be lost after the desired expression cassette comprising the cytokine gene has been transferred to a host genetic element. Typically the vector comprises an origin of replication and a marker by which the vector can be identified and selected (e.g., an antibiotic resistance gene). The cytokine gene may have its own promoter or a different promoter that is capable of driving expression of the coding sequence and that is operably linked to the coding sequence. In either event, he promoter is capable of directing transcription of the coding sequence in the tumor cells.

The cytokine coding sequence can be either genomic or cDNA sequences, and may optionally comprise natural or experimentally induced mutations. Such mutations may lead to a longer half-life, higher expression levels, higher activity, to name just a few possibilities. The modification of the cell line with the additional GM-CSF expression construct is to increase the amount of GM-CSF and to enhance immunogenicity of the tumor cell composition.

The nature of the treatments that are contemplated are in the nature of prophylaxis. Thus a subject with Barrett's esophagus will be treated to prevent or reduce the risk of esophageal cancer. Similarly, a patient with esophageal cancer is treated to prevent or reduce the risk of metastases. Likewise, a patient who has had a resection of an esophageal tumor is treated to prevent or reduce the risk of recurrence.

The propagation-defective cells of the prophylactic compositions can be formulated with pharmaceutically acceptable carriers as are known in the art. Adjuvants may be sued in the formulations. The formulations and/or carriers may be sterile and/or pyrogen-free to reduced risk of infections upon injection.

Inoculations may be my any means known in the art for stimulating an immune response. These may be without limitation, subcutaneous, intramuscular, intravenous, intraperitoneal, intradermal, etc. Suitable dosages can be determined based inter alia on the size and physical robustness of the subject. Single or repeated dosing regimens can be used. Single doses may range from at least 10⁶, 10⁷, 10⁸, or 10⁹ cells. Doses will typically be less than 10¹⁵, 10¹⁴, 10¹³, 10¹², 10¹¹, or 10¹⁰ cells. The repeated dosings can be the same or different preparations, so long as there is an overlapping set of antigens. Dosings can occur at least twice, three times, four times, five times, etc. No maximum number of booster administrations is known.

In order to investigate a novel approach to manage Barrett's esophagus and esophageal cancer, a surgically induced esophageal reflux animal model was utilized⁶⁻⁹. This model promoted in vivo growth of Barrett's esophagus, adenocarcinoma, and adenosquamous carcinoma. Using reflux-associated esophageal tumors harvested from rodents, three cell lines were generated that appeared histologically as squamous cell carcinoma, but retained certain molecular features of adenocarcinoma⁷. The lesions found on the rat esophagi were characteristic of Barrett's esophagus in humans. The rat lesions expressed p53, c-myc, and cyclooxygenase 2, important factors in human carcinogenesis¹⁰. Through cytogenctic analysis, common morphologies, mucin features, and expression of differentiation markers were observed in human and rat esophageal cancers¹¹. Our model has the distinct advantage of promoting rat esophageal tumor growth that resembles human esophageal carcinogenesis. With the availability of an in vivo clinically relevant surgical model, and thus the ability to test new therapies for Barrett's esophagus and esophageal cancer, we proceeded to investigate a tumor vaccine approach to treat esophageal cancer.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

### Materials and Methods for Examples 2-6

All procedures were approved by the Animal Care and Use Committee of Johns Hopkins University and animals received humane care in compliance with the “Guide for Care and use of Laboratory Animals”, published by the National Research Council (National Academy Press, 1996).

### Surgical Model of Reflux Induced Tumors

Biliary reflux was induced in 10 week old, male Sprague Dawley rats, (200-250 gms, Harlan, Indianapolis, Ind.). The animals were allowed two weeks to acclimatize, and were housed at temperatures of 20-22° C., humidity of 30-50% at 12 hour alternating light-dark cycles. The rats were fasted overnight, but were allowed water ad-libitum until the surgery. The rats were anesthetized with an intramuscular injection of ketamine and xylazine. The esophagus was mobilized preserving the vagus nerves, and the loop of jejunum was then identified 4 cm from the ligament of Treitz. The gastroesophageal junction was divided and ligated, and an end-to-side anastomosis was constructed between the distal esophagus and jejunum (modified Levrat's model)²¹. Once the animals awoke, they were allowed water ad-libitum, and feeding recommenced the following day. Animals received appropriate analgesia during the peri-operative and postoperative periods. The experiment concluded 9 months post-operatively.

### Tumor Harvest

Animals were anaesthetized in a manner as previously described. Abdominal and thoracic viscera were exposed through a midline incision, and tumors were assessed for size, and extent. The rat's esophagi were mobilized from the neck down to and including the jejuno-esophagostomy. Tumors were identified and carefully dissected away from the liver and adjoining structures. Esophagi were opened longitudinally to expose the lumen. A small representative portion of tissue was excised from the tumor mass and preserved in 10% formalin for histological examination. Harvested tumors were washed immediately with phosphate buffered saline (PBS) solution to remove any food debris and contaminants. The samples were stored in PBS at 4° C. and transported to the laboratory for use in tissue culture.

Animals were euthanized with 150 mg/kg of Pentobarbital by intracardiac injection and cervical dislocation under anesthesia.

### Establishment of the Cell Lines

Esophageal tumors were excised from rats 9 months post operation. The harvested rat tumors were mechanically minced and enzymatically digested using collagenase. The disaggregated cells were plated in appropriate growth media as primary cultures. The culture plates were checked for the presence of cancer cells every day. After adequate growth, a single clone was picked using a cloning ring and transferred to 96 well plates. Aliquots of the original lineage were frozen and stored. The tumor cells displayed the ability to continue to undergo (over 40) passages without changing phenotype¹¹.

### Flow Cytometry

Cell line expression of major histocompatibiltity complex (MHC) class I molecules was assessed using flow cytometry. The cells were stained with 10 μl of mouse MHC class I monoclonal antibody (clone OX-18, Abcam, Cambridge, Mass.) per 10⁶ cells for 40 minutes at 4° C. The samples were washed and analyzed on a Becton, Dickenson and Company LSR Flow Cytometer (Franklin Lakes, N.J.) at the SKCC Cell Imaging Core of the Johns Hopkins University School of Medicine (Baltimore, Md.).

### Creation of Tumor Vaccine

We used electroporation to transfect a DNA expression vector, pCDNA3 (CONTROL) or mGM-pCDNA3.1 (with GM-CSF), from Invitrogen Life Technologies, Carlsbad, Calif., into our tumor cells. The transfected tumor cells GM-CSF secretion was assessed using a Quantikine ELISA kit (R&D Systems, Minneapolis, Minn.). Transfected GM-CSF secreting vaccine cells and non-transfected tumor cells were irradiated with 5000 Gy prior to injection to prevent their propagation.

### Animal Vaccination

The rodents were divided into two treatment groups and immunologic responses at the injection sites were assessed. Animals were injected with either 1×10⁷ cells/0.5 ml of irradiated tumor cells secreting GM-CSF (+) or an equivalent injection of non-transfected GM-CSF (−) irradiated tumor cells into all four limbs.

### Tumor Transplants

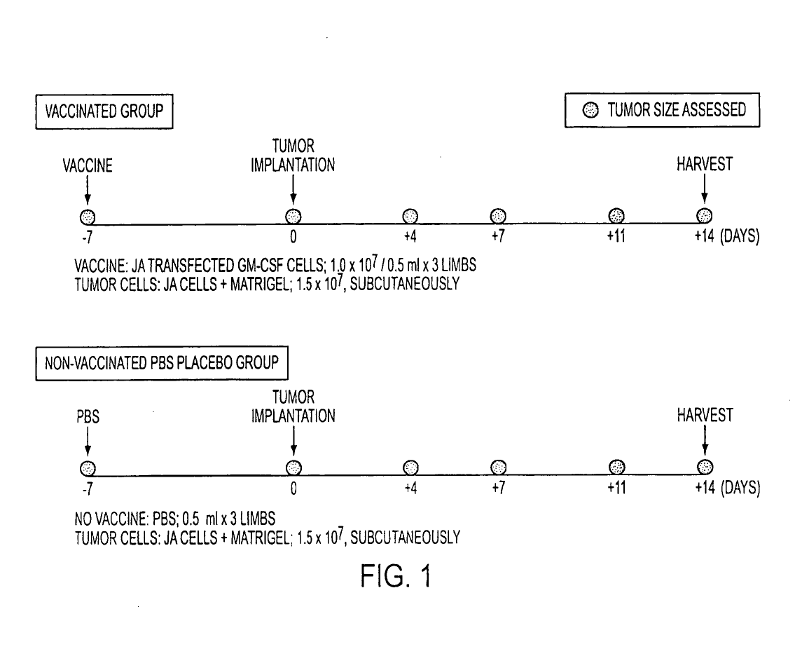

Transplantable tumors were injected at a dose of 1.5×10⁷ cells with 0.1 mL of Matrigel™ (BD Biosciences, San Jose, Calif.) into the subcutaneous tissue of the back. The rats were either administered 1×10⁷ cells/0.5 ml of irradiated tumor cells secreting GM-CSF (+) or an equivalent amount of PBS into both upper limbs and the lower left limb according to the schedule described in FIG. 1. Injection sites were biopsied and assessed on days 3, 5, 7, and 14. The sizes of transplanted tumors were measured and plotted graphically to assess tumor growth on day 0, 4, 7, 11, and 14. Tumor volume was calculated according to the following formula: (a×b²)/2, where a is the larger and b is the smaller of the dimensions.

### Rat Surgical Reflux Model

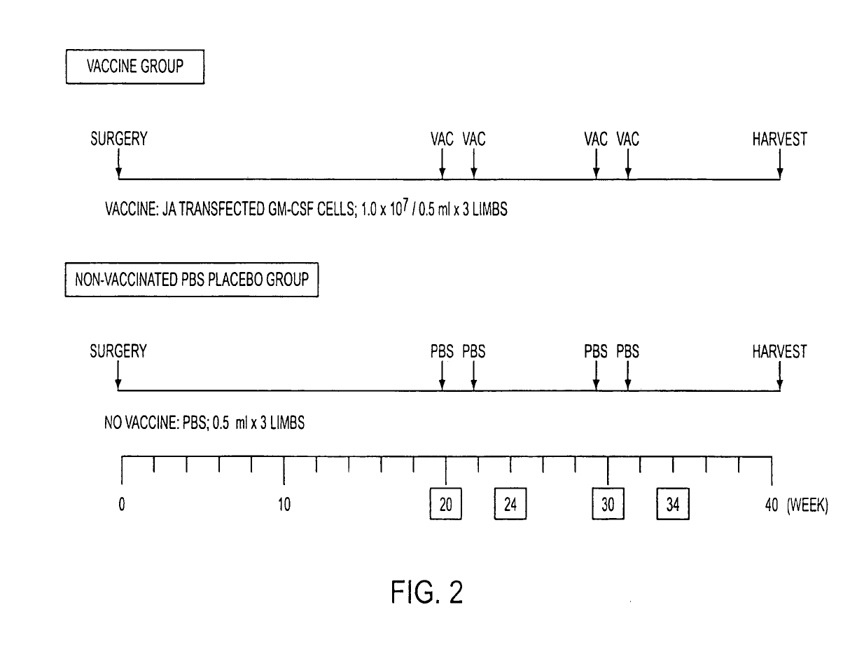

The rat reflux model was created by performing a jejuno-esophagostomy on Sprague Dawley rats, as established by Miwa et al. in 1996 9. Sixty two animals were randomly divided into two groups: a vaccine group (n=22) and a PBS non-vaccinated PBS placebo group (n=38). Rats were vaccinated 20, 24, 30, and 34 weeks after surgery with 3×107 irradiated vaccine cells secreting GM-CSF or an equivalent amount of PBS as shown in FIG. 2. All rats were weighed every 4 weeks throughout the experiment. All surviving animals were sacrificed 40 weeks after surgery and their esophagi were examined.

### Tissue Preparation

Implantation Experiment

Implanted tumors (including skin) and tissue from the injection site were harvested, cut in half, frozen and stored at −70° C. for staining for CD4⁺ and CD8⁺ T cells. The remaining samples were fixed in 10% formalin for 24 hours and embedded in paraffin for hematoxylin and eosin. Microscopy of five random samples was used to estimate the size of granulomas. The number of eosinophils in each of the random samples was estimated using high power field microscopy. Results were recorded as the percentage of eosinophils/total number of cells×100.

Reflux Model Experiment

The thyroid cartilage and the site of anastomosis were used as landmarks during the resection of the esophagus and jejunum. The specimen was cut longitudinally into three 1 mm wide slices of the esophageal mucosa. The slices were fixed in 10% formalin for 24 hours and then embedded in paraffin for Hematoxylin and Eosin (H&E) staining.

### Immunohistochemistry

Immunohistochemical staining with specific antibodies was used to determine the localization of CD4+ and CD8+ cells. The frozen 5 μm tissue sections were immersed in absolute methanol containing 0.3% hydrogen peroxidase, and then covered with normal goat serum (diluted 1:50). Sections were then incubated overnight at 4° C. with a primary antibody to rat CD4 and CD8a, diluted 1:50. (BD Biosciences, San Jose, Calif.) and sections were treated with a labeled polymer (DAKO, CA, USA) for 2 hours.

Reaction products developed following the immersion of a section into 3,3′-diaminobenzil tetradrochloride. The slides were counterstained lightly with hematoxylin. High power field microscopy was used to determine the count of cells positive for CD4 and CD8. Results were expressed as the total number of positive cells/total number of cells×100.

### Pathological Assessment

Pathological assessment was carried out on 5 μm H&E stained sections from each block. The pathological progression due to duodeno-esophageal reflux was categorized as one of the following:

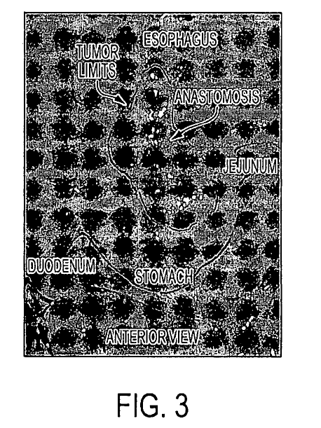

Proliferative squamous hyperplasia: Proliferative hyperplasia is a condition characterized by a thickened epithelium to twice that of a normal epithelium with acanthosis, elongation of the papillae, and parakeratosis. Other features include the thickening of the basal layer of the squamous epithelium and the preservation of a stratified appearance (FIG. 3A).

Barrett's metaplasia: Barrett's metaplasia is defined by the presence of columnar-lined epithelium with intestinal metaplasia replacing the esophageal squamous epithelium (FIG. 3B).

Carcinoma: Epithelial growth with cellular and structural atypia, invading into the submucosal layer was defined as carcinoma. Adenocarcinoma consists of malignant-appearing glandular cell growth with both atypia and invasiveness that has two types of histology: tubular adenocarcinoma and mucinous adenocarcinoma (FIG. 3C). Squamous cell carcinoma is a well differentiated carcinoma marked by cellular and structural atypia and squamous pearls (FIG. 3D).

### Statistical Analysis

All quantitative studies were expressed as the mean±standard error (SE). Differences were analyzed for significance using one-way ANOVA, Student's t test or Fischer's exact test, as appropriate. Data management and statistical analysis were performed using Stat View software (SAS Co., Berkeley, Calif.). Data values were considered significant when the p value was <0.05.

### Example 2

### Establishment of In Vivo Tumors

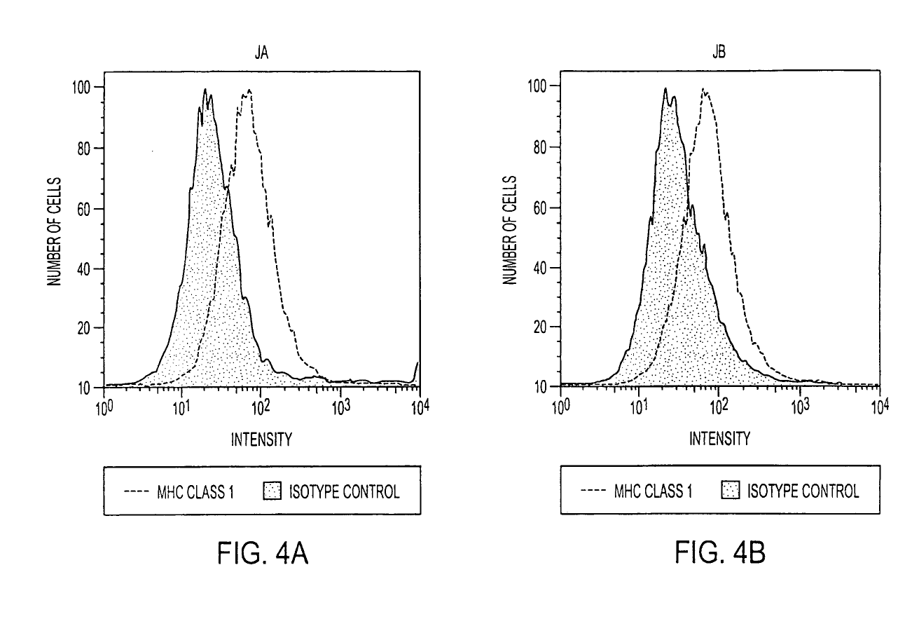

We utilized a surgically-induced esophageal reflux model⁷(FIG. 4). The model produced pre-malignant lesions of Barrett's esophagus and progressed through dysplasia to carcinoma of the esophagus. The findings reported by other groups were similar to the results of our own vaccine study^{8,9,22-26}.

In our model, 75% of the animals developed Barrett's metaplasia, and 50% progressed to adenocarcinoma. A small proportion of tumors displayed characteristics of squamous cell carcinoma with keratin pearls. This model had the advantage of producing both Barrett's metaplasia as well as esophageal carcinoma, which were the potential targets of our vaccine. All cell lines were generated from tumors extracted from fields of Barrett's metaplasia and displayed characteristics of adenocarcinoma²⁷.

The in vitro propagation of two resected cancers that developed as a result of the rat reflux surgical model were used to create three cell lines—the JA and JB lines from one case, and the AMY cell line from the other. We demonstrated the ability of these cell lines to propagate long-term in vitro and to form tumors in vivo (i.e., xenografts) in athymic mice as proof of their malignant character⁷.

Aliquots from the original cells were passaged up to 40 times without any morphological change in the three cell lines. The cytospin histology of the JA and JB cell lines showed well differentiated squamous cell carcinoma, while the cytospin of the AMY cell line displayed minor presence of mucinous cells.

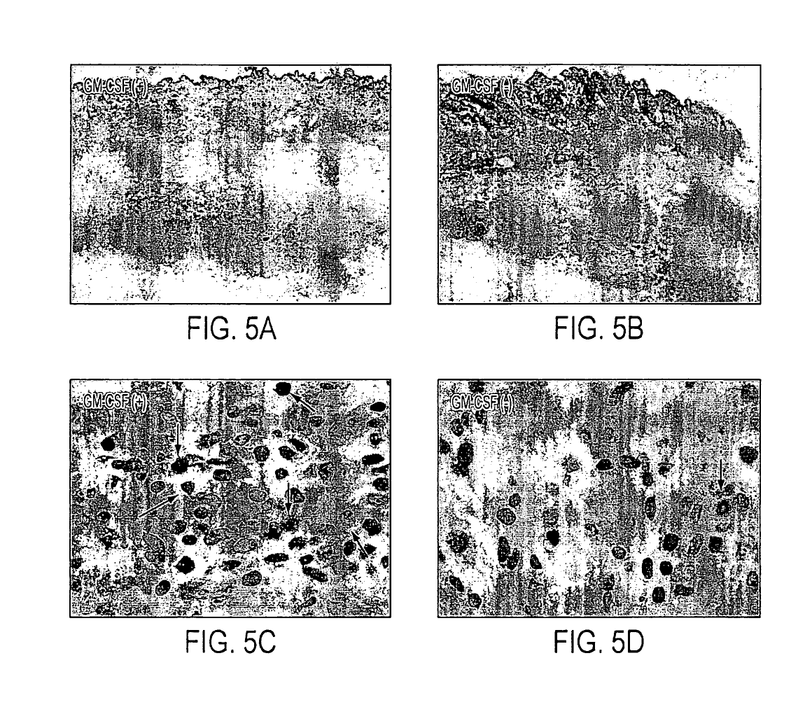

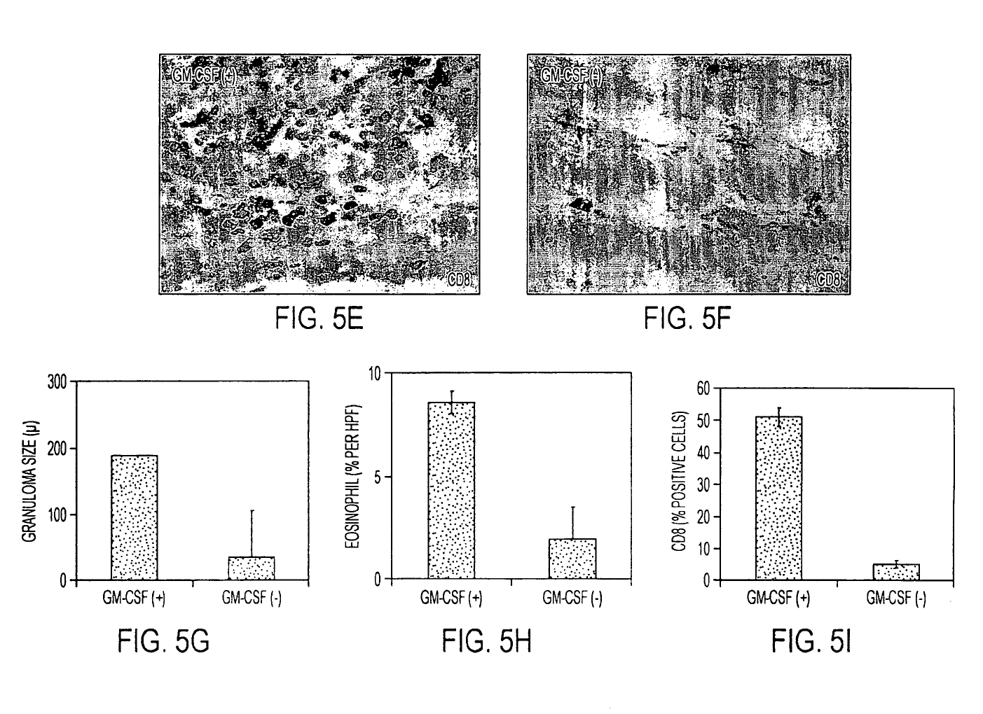

In order to be effectively rejected by the T-cell arm of the immune system, tumors must express the MHC class I complex. Flow cytometry was used to assess the expression of MHC class I molecules in our cell lines. While both the JA and JB cell lines expressed MHC class I molecules, the AMY cell line did not (FIG. 5).

Tumor cells from the JA and JB cell lines were transfected to express GM-CSF, thereby creating our tumor vaccine. A GM-CSF Enzyme Linked ImmunoSorbent Assay (ELISA) was used to test whether the transfection was successful. The JA cell's GM-CSF secretion was confirmed at 55 ng/10⁶ cells/24 hours, which was greater than the JB cell's. Therefore, the JA cell line was chosen to be used in the tumor vaccine. Prior to utilizing the cells for our vaccine, they were irradiated with 5000 Gy to prevent further propagation.

### Example 3

### Immunologic Response to the Vaccine at the Injection Site

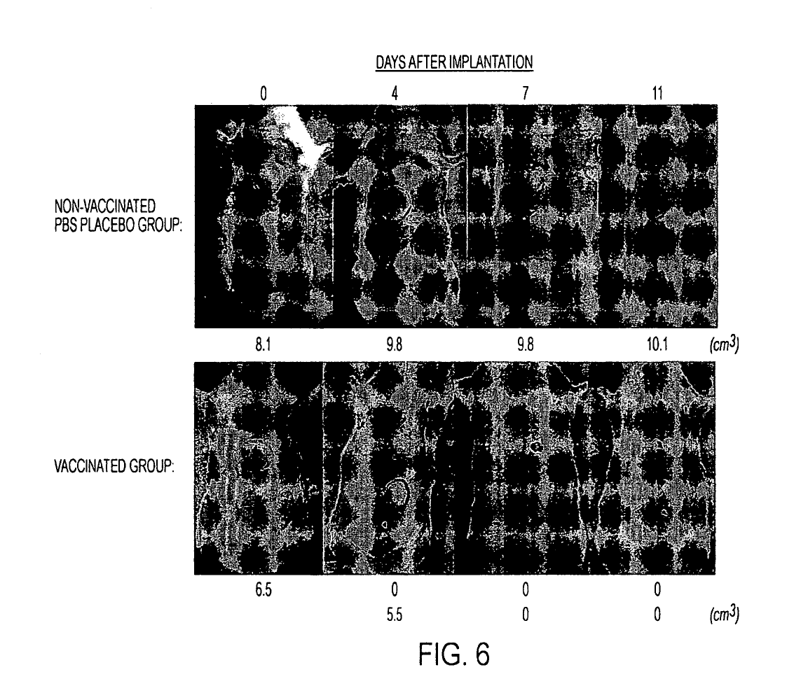

GM-CSF secreting JA cells were found to promote an inflammatory response at the vaccination site. The infiltration of T-cells, eosinophils, macrophages, and the granuloma size were statistically greater in rats vaccinated with GM-CSF cells than irradiated JA cells that had not been transfected to express GM-CSF. Immunohistochemical staining showed that vaccine cells, which were irradiated and transfected with the GM-CSF expression vector promoted the infiltration of CD8⁺ T-cells into the vaccine-injected site. This was greater than the cells, which were not irradiated and transfected with the GM-CSF vector (FIG. 6).

### Example 4

### Efficacy of a Vaccine in a Rat Tumor Transplant Model

Tumor transplants derived from the JA cell line were established in rats. The rats were vaccinated 7 days prior to tumor transplantation according to the schedule described previously, and tumor growth was assessed sequentially 0, 4, 7, and 11 days following transplantation. All animals were sacrificed 14 days following transplantation.

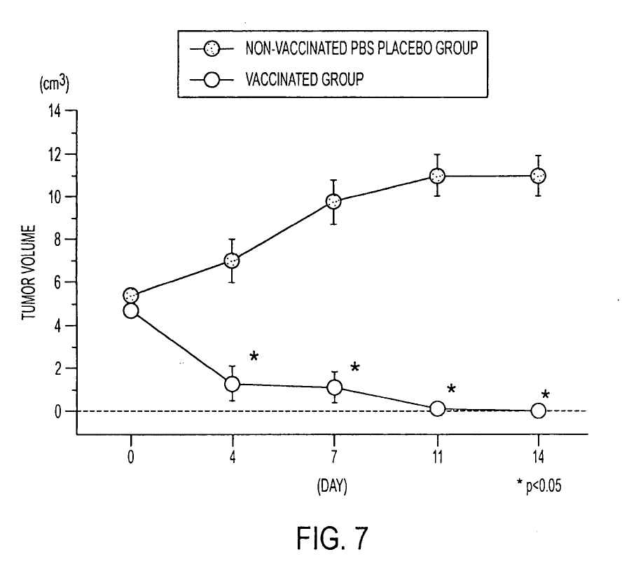

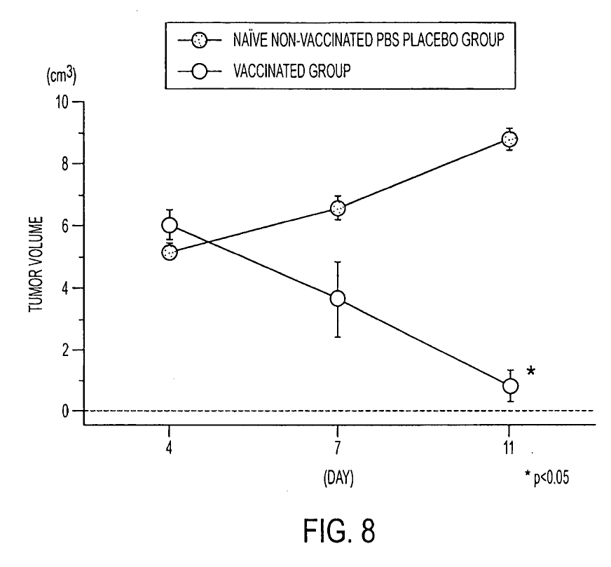

Rodents vaccinated with GM-CSF secreting tumor cells displayed significant tumor regression within 4 days following transplantation (FIG. 7). On day 4, four of the six animals in the vaccination group no longer displayed tumors, and the tumors present in the remaining two animals disappeared by day 14. At the time of sacrifice or day 14, no tumors were present in any of the vaccinated animals (FIG. 8). However, tumors on rats belonging to the non-vaccinated PBS placebo group grew in size at a constant rate until they were sacrificed.

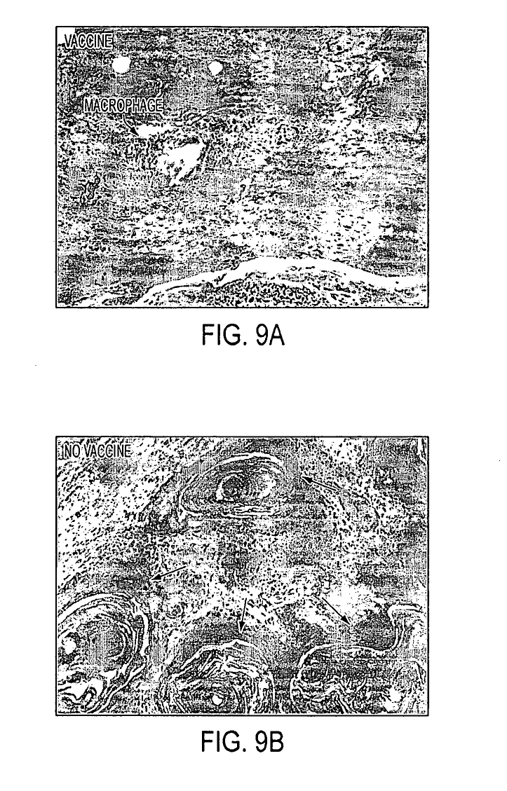

To assess the magnitude of response to the vaccination, four animals whose tumors disappeared by day 4 were re-transplanted with additional tumor cells on day 4. In addition, four naïve animals were transplanted for the first time and served as the naïve non-vaccinated PBS placebo group. In order to follow a protocol similar to the original transplantation, tumor size was assessed on day 11 or one week post re-transplantation. Re-transplanted vaccinated animals observed a significant decrease in tumor size, while tumors on naïve non-vaccinated PBS placebo group rodents grew in size at a rate similar to those in the original non-vaccinated PBS placebo group (FIG. 9).

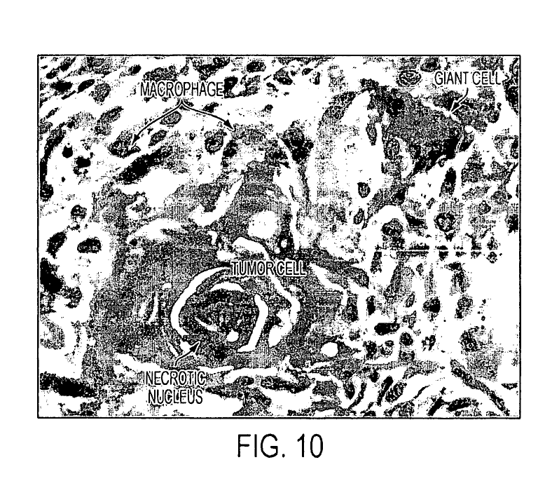

A histological assessment of the transplant site, which was harvested on either day 14 for animals belonging to the non-vaccine and vaccine groups, or day 11 for the animals belonging to the naïve non-vaccinated and re-transplantation groups, was evaluated by H&E. The non-vaccinated animals exhibited tumor growth. An abundance of neoplastic cells and keratin pearls were seen in the naïve non-vaccinated PBS placebo group and in the non-vaccinated PBS placebo group. On the other hand, the vaccinated animals displayed macrophages, lymphatic infiltrate, and dead or dying tumor cells (FIG. 10). This confirmed the efficacy of the vaccine against tumors in the re-transplantation group as well as in the original implantation group.

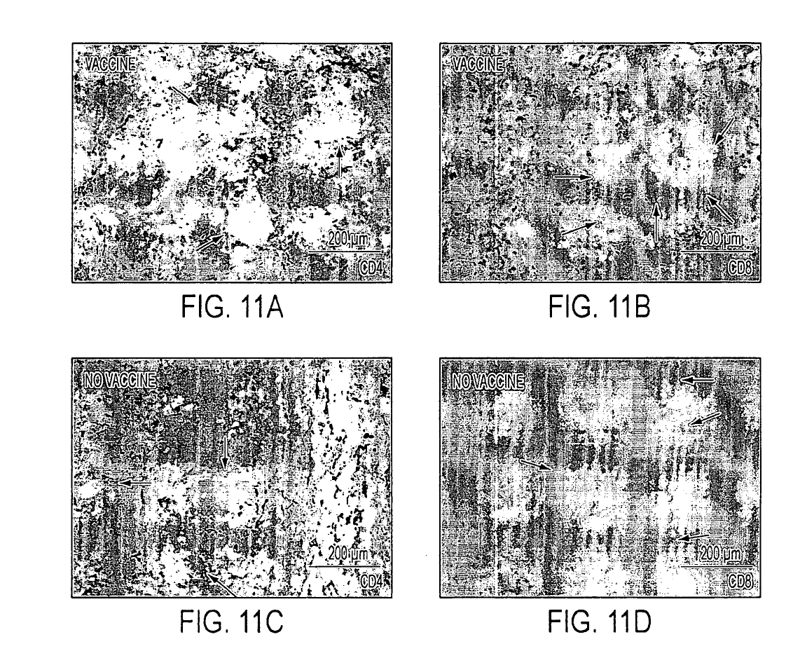

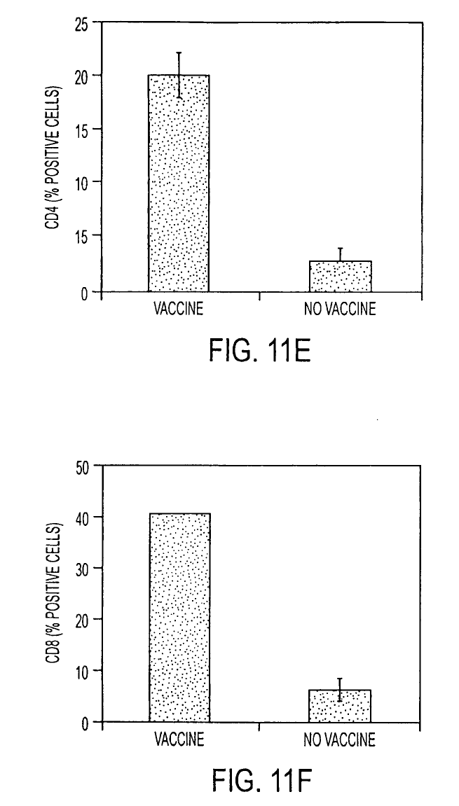

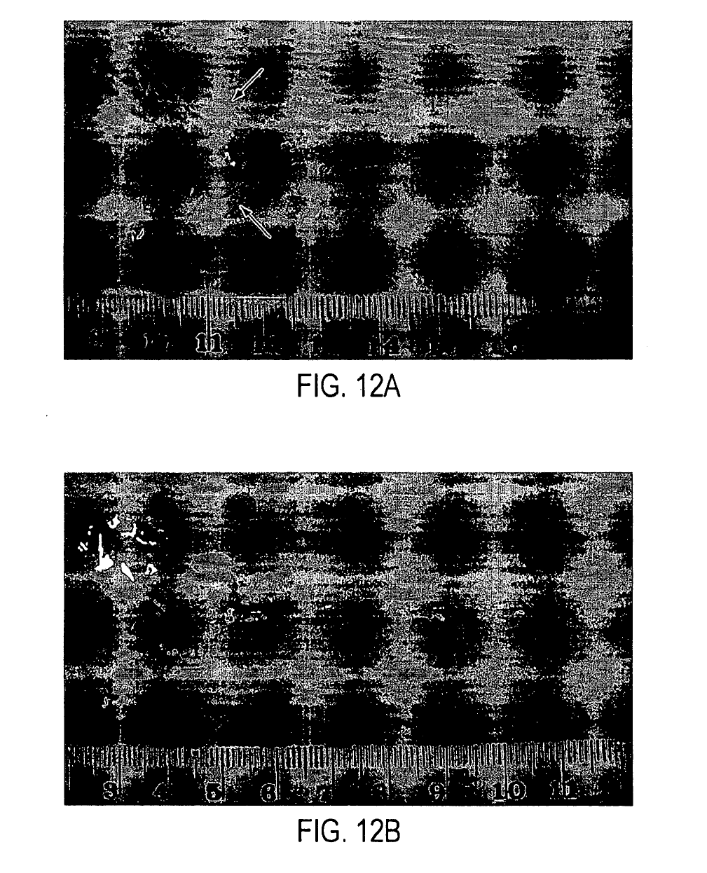

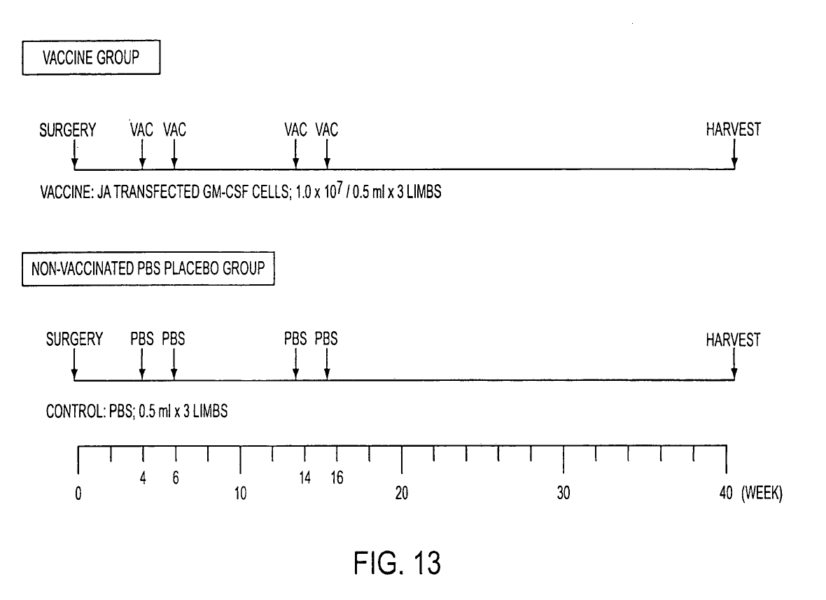

Macrophages and giant cells in the vaccinated groups completely surrounded and destroyed tumor cells and necrotic nuclei (FIG. 11).

Immunohistochemical staining showed that tumors implanted in animals vaccinated with irradiated cells transfected with GM-CSF expression vector, promoted tumor infiltration of CD4⁺ T-cells and CD8⁺ T-cells. This infiltration was minimal in animals that were not vaccinated, but injected with PBS as a mock vaccine (FIG. 12).

### Example 5

### Rat Surgical Reflux Model

Of the 60 operated animals, 39 rats survived 40 weeks post-surgery and were included in the study. Of these, 23 were part of the non-vaccinated PBS placebo group, while the remaining 16 were subjected to a vaccine. A total of 21 rats (15 in the PBS non-vaccinated PBS placebo group and 6 in the vaccine group) died of surgical complications, such as malnutrition, pneumonia, and unknown causes. No differences in mortality were noted between the two groups. Body weight did not differ between the two groups (Table 1).

TABLE 1
Histopathological Findings

| | Non-vaccinated PBS | |
|---|---|---|
| | placebo group | Vaccine group |
| Total Surgeries | 38 | 22 |
| Survivors | 23 | 16 |
| Average Weight +/− Std. | 292 +/− 58 | 318 +/− 55 |
| Error | | |
| Cancer | 17 (74%)* | 6 (38%)* |
| Adenocarcinoma | 14 | 6 |
| Squamous Cell Carcinoma | 3 | 0 |
| Barrett's Metaplasia | 23 (100%) | 13 (81%) |
| Proliferative Hyperplasia | 23 (100%) | 16 (100%) |

The incidence of cancer was lower in the vaccine group, as compared to the non-vaccinated PBS placebo group (*p < 0.05).

Macroscopic

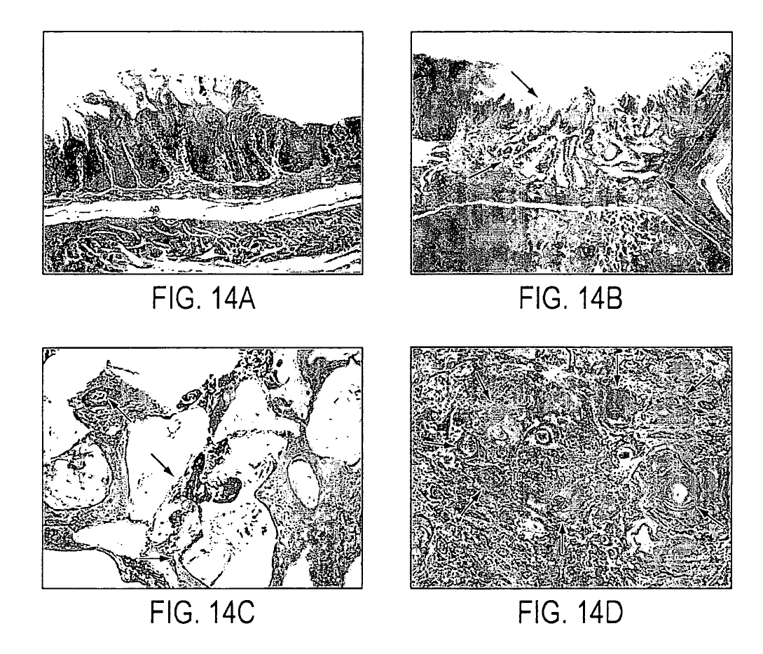

In the non-vaccinated PBS placebo group, the middle to distal portions of the esophagi were thickened in all rats and the epithelia were irregular (FIG. 14A). In contrast, the rats in the vaccine group displayed smooth distal portions of the esophagus with normal width (FIG. 14B).

Microscopic

While 74% ( 17/23) of animals in the non-vaccinated PBS placebo group developed esophageal cancer, animals in the vaccine group had an incidence of cancer of 38% ( 6/16). Animals in the vaccine group had a lower chance of developing cancer than the placebo group (p<0.05) (Table 1). In the placebo group, 14 rats displayed adenocarcinoma and three rats developed squamous cell carcinoma, while six rats in the vaccine group developed adenocarcinoma. The rats in the vaccine and placebo groups developed proliferative hyperplasia. Barrett's metaplasia was found on 100% ( 23/23) of the rats in the placebo group, but there was a protective tendency in the vaccinated group with 81% ( 13/16) of the rats displaying signs of Barrett's metaplasia.

### Example 6

Esophageal cancer is a disease with increasing incidence and morbidity. In order to combat the disease, we proposed to use innovative molecular approaches. Stable tumor cell lines for use in in vitro and in vivo studies were derived from reflux induced tumor cells in rodents. Established cell lines were used to create a whole-cell, GM-CSF secreting tumor vaccine for use in the treatment of esophageal cancer in rats. The vaccine was prepared by engineering tumor cells to stably express GM-CSF, a cytokine that has displayed promise in other cancer vaccine studies²⁸. Following successful transfection and prior to injection into rodents, tumor cells were irradiated to prevent further propagation.

The efficacy of the GM-CSF secreting vaccine was tested using a tumor transplant model. This model was used for convenience and proof of principle. Transplanted tumors have the distinct experimental advantage of growing quickly, such that data is rapidly available and hypotheses may be tested efficiently. The subcutaneous tissue of rats was chosen as the location for tumor transplantation, because the site offers distinct advantages. First, the rat's immune system is given the opportunity to illicit a strong and uniform response against the tumor. Secondly, the transplantation site allowed experimenters to visualize and measure tumor sizes over time.

Pre-implantation vaccination inhibited tumor growth and resulted in histologic changes consistent with immunologic infiltration and tumor cell death. The transplanted tumors decreased in size and disappeared in rats that were administered a vaccination. Microscopic studies supported these macroscopic findings. The results of immunohistochemical studies confirmed the vaccine promoted the recruitment of CD8⁺ and CD4⁺ T-cells to the vaccine and tumor sites. These findings allow us to eliminate tumor over growth and necrosis as a potential cause of the statistically significant decrease in tumor size between the vaccinated and non-vaccinated groups. To further confirm the robust immune response generated in vaccinated animals, tumors were re-transplanted in rats whose primary transplanted tumors had disappeared by day 4. The immune systems of rats re-transplanted with secondary tumors were able to once again successfully impede tumor growth better than rats not administered a vaccine. These results were confirmed macroscopically and microscopically. Histological assessments on day 11, the day that the re-implanted secondary tumor disappeared, indicated the presence of a significant number of CD8⁺ cells. Tissue samples from the injection site of vaccinated rats displayed increased numbers of granulomas and eosinophils compared to samples from non-vaccinated placebo rats. Therefore, the most likely explanation of our findings is that the vaccine successfully induced an antitumor immune response.

We confirmed these results in a clinically relevant surgical reflux model. In a prior study, we aimed to use the vaccine to prevent the development of duodeno-esophageal reflux induced Barrett's esophagus. In a similar rat surgical model, Barrett's was shown to develop 20 weeks post-surgery²⁹. We vaccinated rats prior to the development of Barrett's at 4, 6, 14, and 16 weeks following surgery. The present study was performed to investigate the effectiveness of the vaccine to impede the progression of Barrett's to cancer. All rats underwent a jejuno-esophagostomy. All vaccinations were administered after the development of Barrett's or 20 weeks post-surgery. Histopathological findings confirmed that regardless of treatment, nearly all rats displayed signs of Barrett's esophagus. Operated rats administered a GM-CSF vaccine developed cancer at a lower rate than non-vaccinated PBS placebo rats injected with an equivalent amount of saline (p<0.05). These results suggest the strong potential of use of our vaccine as a future treatment option for gastro-esophageal reflux disease associated esophageal cancer.

The vaccine used in these studies was generated from the JA cell line, which expresses a squamous phenotype when transplanted into nude mice⁷. It may seem surprising that the vaccine made with the JA cells could protect against the development of carcinoma in the reflux model where the phenotype includes adenocarcinoma in addition to squamous cancer. However, our cytogenetic analysis shows important similarities between rat and human esophageal cancers. Through cytogenetic analysis we found a highly aneuploid cell population with the derangement of key chromosomes that encode for a variety of oncogenes. The neoplastic nature and gene expression profile of these rodent reflux induced tumors was found to be comparable to human esophageal cancer⁷. The Y chromosome is deleted and translocation of chromosome 7 and 11 occurs as does over expression of important peptide mediators thought to be important in carcinogenesis, including hypoxia-inducible factor-1 alpha, cyclin dependent kinase 4, vascular endothelial growth factor, polo-like kinase, and the epidermal growth factor receptor⁷. These and other as yet unrecognized cellular similarities between the JA cell lines and adenocarcinoma, may explain the effectiveness of the JA whole cell vaccine in protecting against the development of adenocarcinoma in the rat model. Therefore, the rat model can be appropriately used to study the processes involved in esophageal carcinogenesis in humans. For further reference, the differentially expressed genes are deposited at the NCBI's Gene Expression Omnibus and are accessible through the GEO series accession number GSE1707. There are at least two well known human esophageal adenocarcinoma cell lines, namely BIC and SEG. These cell lines would be candidates for use in a future human trial. Further studies will allow us to test whether antigens are seen by the immune system.

Potential targets for the esophageal cancer vaccine would be patients with dysplastic Barrett's. The vaccine could be given as a preventative strategy to impede the development of cancer from dysplastic Barrett's, thereby reducing the dependence on invasive techniques such as an esophagectomy, and other ablative modalities. Another potential use of this vaccine is as an adjuvant therapy for individuals who have had potentially curative surgical resection of their tumors. In both of these scenarios the patients' tumor burden would be small at the time of vaccination, so the vaccine would not have to destroy a large mass of tumor. It would rather prevent tumor growth, which seems to us to be an appropriate role for the vaccine approach. Clearing large, established tumors seems to be a particularly unlikely role for a tumor vaccine. The potential role of the vaccine for treating dysplastic Barrett's is especially attractive as these patients will not have developed immune tolerance to esophageal cancer. In this setting, the vaccine would function in a purely preventive mode.

In summary, we were the first to develop a whole-cell GM-CSF vaccine for esophageal cancer in animals. We were able to generate a stable esophageal cancer cell line. A GM-CSF secreting cancer vaccine derived from these cells successfully inhibited further cancer growth in subcutaneously transplanted tumors and carcinogenesis in a surgical rat reflux model. This vaccine has potential as a preventive or treatment modality for combating esophageal cancer.

### Example 7

### Materials and Methods for Example 8

### Animal Vaccination

Vaccine cells were irradiated with 5000 Gy to prevent their propagation. Animals were injected with 1×10⁷ cells/0.5 ml of irradiated tumor cells secreting GM-CSF or an equivalent amount of phosphate buffer solution (PBS) into both the upper limbs and the lower left limb. The rats were vaccinated according to the schedule described in FIG. 1.

### Rat Surgical Reflux Model

The rat reflux model was created by performing a total gastrectomy on Sprague Dawley rats, followed by a jejuno-esophago-anastomosis, as established by Miwa et al. in 1996 (Miwa, Sahara et al. 1996). Fifty four animals were randomly divided into two groups: a vaccine group (n=30) and a PBS non-vaccinated PBS placebo group (n=24). Rats were vaccinated 4, 6, 14, and 16 weeks after surgery with 3×107 irradiated vaccine cells secreting GM-CSF or an equivalent amount of PBS as shown in FIG. 1. All rats were weighed every 4 weeks throughout the experiment. All surviving animals were sacrificed 40 weeks after surgery and their esophagi were examined.

### Tissue Preparation

The thyroid cartilage and the site of anastomosis were used as landmarks during the resection of the esophagus and jejunum. The specimen was cut longitudinally into three 1 mm wide slices of the esophageal mucosa. The slices were fixed in 10% formalin for 24 hours and then embedded in paraffin for Hematoxylin and Eosin (H&E) staining.

### Pathological Assessment

As previously described, pathological assessment was carried out on 5 μm H&E stained sections from each block (Miyashita, Armstrong et al. 2007). The pathological progression due to duodeno-esophageal reflux was categorized as one of the following:

Proliferative squamous hyperplasia: Proliferative hyperplasia is a condition characterized by a thickened epithelium to twice that of a normal epithelium with acanthosis, elongation of the papillae, and parakeratosis. Other features include the thickening of the basal layer of the squamous epithelium and the preservation of a stratified appearance (FIG. 2A).

Barrett's metaplasia: Barrett's metaplasia is defined by the presence of columnar-lined epithelium with intestinal metaplasia replacing the esophageal squamous epithelium (FIG. 2B).

Carcinoma: Epithelial growth with cellular and structural atypia, invading into the submucosal layer was defined as carcinoma. Adenocarcinoma consists of malignant-appearing glandular cell growth with both atypia and invasiveness that has two types of histology: tubular adenocarcinoma and mucinous adenocarcinoma (FIG. 2C). Squamous cell carcinoma is a well differentiated carcinoma marked by cellular and structural atypia and squamous pearls (FIG. 2D).

### Statistical Analysis

A Fischer's exact test was used for statistical analysis on the incidence of pathological findings. Data management and statistical analysis was performed using Stat View software (SAS Co., Berkeley, Calif.) and differences were considered significant when the p value was <0.05.

### Example 8

### Rat Surgical Reflux Model

Of the 54 operated animals, 32 rats survived 40 weeks post-surgery and were included in the study. Of these, 16 were part of the non-vaccinated PBS placebo group, while the remaining 16 were subjected to a vaccine. A total of 22 rats (8 in the PBS non-vaccinated PBS placebo group and 14 in the vaccine group) died of surgical complications, such as malnutrition, pneumonia, and unknown causes. No differences in mortality were noted between the two groups. Body weight did not differ between the two groups (Table 2).

TABLE 2
Histopathological Findings

| | Non-vaccinated PBS | |
|---|---|---|
| | placebo group | Vaccine group |
| Total Surgeries | 24 | 30 |
| Survivors | 16 | 16 |
| Average Weight +/− Std. | 272 +/− 81 g | 273 +/− 73 g |
| Error | | |
| Cancer | 15 (94%)* | 4 (25%)* |
| Adenocarcinoma | 14 | 4 |
| Squamous Cell Carcinoma | 1 | 0 |
| Barren's Metaplasia | 16 (100%) | 13 (82%) |
| Proliferative Hyperplasia | 16 (100%) | 16 (100%) |

The incidence of cancer was significantly lower in the vaccine group, as compared to the non-vaccinated PBS placebo group (*p < 0.05).

### Macroscopic

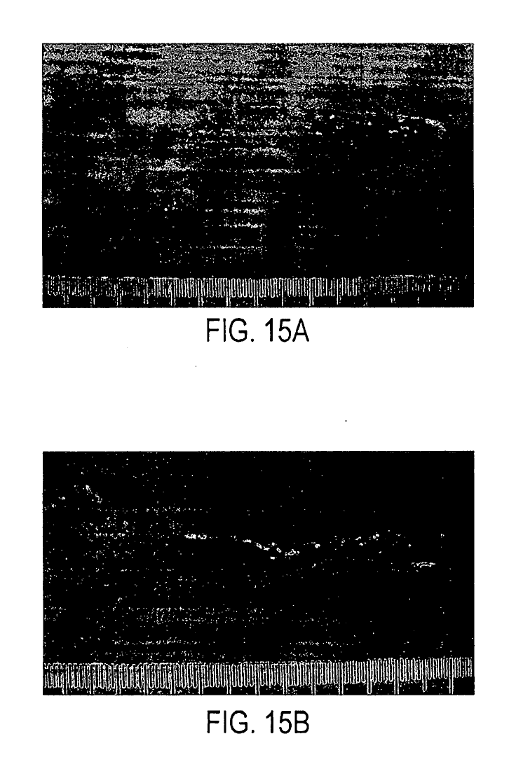

In the non-vaccinated PBS placebo group, there was pronounced thickening of the esophagi in all rats. The esophageal epithelia displayed areas of irregularity (FIG. 15A). On the other hand, vaccinated rats displayed smooth distal portions of the esophagus with normal width (FIG. 15B). These results are similar to our previously reported findings (Miyashita, Armstrong et al. 2007).

### Microscopic

While 94% ( 15/16) of animals in the non-vaccinated PBS placebo group developed esophageal cancer, animals in the vaccine group had an incidence of cancer of 25% ( 4/16). Animals in the vaccine group had a significantly lower chance of developing cancer than the placebo group (p<0.05). In the placebo group, 14 rats displayed adenocarcinoma and one rat developed squamous cell carcinoma, while four rats in the vaccine group developed adenocarcinoma. The rats in the vaccine and placebo groups developed proliferative hyperplasia. Barrett's metaplasia was found on 100% ( 16/16) of the rats in the placebo group, but there was a protective tendency in the vaccinated group with 82% ( 13/16) of the rats displaying signs of Barrett's metaplasia.

### Example 9

We have created an animal model of esophageal cancer that enabled the establishment of stable tumor cell lines for use in in vitro and in vivo studies of this devastating disease (Bonde, Sui et al. 2007). From this model we created a whole-cell, GM-CSF secreting tumor vaccine for the prevention of Barrett's and esophageal cancer in clinically relevant animal surgical models. To create the vaccine tumor cells were transfected using electroporation to stably express GM-CSF. Those cells were irradiated and used to vaccinate animals.

We have confirmed the efficacy of the vaccine against esophageal carcinogenesis when it is administered after the development of Barrett's. In a previous study, we transplanted tumors subcutaneously in rats vaccinated with the GM-CSF secreting whole cell vaccine used in this experiment (Miyashita, Armstrong et al. 2007). The vaccine produced inflammatory infiltrates, granulomas, and eosinophils at the vaccination site. Histological examinations of the implantation site showed infiltration with CD4⁺ and CD8⁺ T cells around the tumor and tumor cell death. As seen through a high power field microscope, the vaccine recruited granulomas, eosinophils, and CD8⁺ T cells at the injection site. The immune response effectively impeded tumor growth at the implantation site. We confirmed in a clinically relevant surgical reflux model the results we observed in our prior subcutaneous tumor transplant study. Rats vaccinated after the development of Barrett's developed cancer at a lower rate than rats administered an equivalent injection of saline (p<0.05) (Miyashita, Armstrong et al. 2007).

In the present study, we aimed to determine the efficacy of our vaccine when it is administered before the development of Barrett's esophagus. All rats underwent a total gasterectomy, followed by an esophago-jejunostomy. As reported previously, rats subjected to this surgical model develop Barrett's 20 weeks post-surgery (Miyashita, Ohta et al. 2006). Animals were vaccinated or injected with a saline equivalent prior to the development of Barrett's or 4, 6, 14, and 16 weeks post-surgery. Regardless of treatment, nearly all rats displayed signs of Barrett's esophagus. The vaccine was not effective at preventing the development of Barrett's. However, the vaccine impeded the progression of Barrett's to cancer. These results suggest the potential of use of our vaccine as a preventive option for Barrett's associated esophageal cancer.

In considering the application of this type of vaccine in humans it is important to consider the similarities between rat and human esophageal cancers. Through cytogenetic analysis we found a highly aneuploid cell population with the derangement of key chromosomes that encode for a variety of oncogenes. The neoplastic nature and gene expression profile of these rodent reflux induced tumors was found to be comparable to human esophageal cancer (Bonde, Sui et al. 2007). The expression levels of epidermal growth factor, vascular endothelial growth factor, polo like factor, and the deletion of chromosome 17 were all similar to findings observed in human esophageal cancer (Bonde, Sui et al. 2007). Therefore, the rat model can be appropriately used to study the processes involved in esophageal carcinogenesis in humans. For further reference, the differentially expressed genes are deposited at the NCBI's Gene Expression Omnibus and are accessible through the GEO series accession number GSE1707.

This project was the first to develop a successful whole-cell GM-CSF esophageal cancer vaccine for rodents. Other investigators have successfully developed whole cell vaccines and begun early clinical trials for pancreatic (Jaffee, Hruban et al. 2001), breast (Emens, Armstrong et al. 2004), renal (Simons, Jaffee et al. 1997), prostate cancers (Simons, Mikhak et al. 1999), and melanoma (Soiffer, Lynch et al. 1998). It is clear from our experience with the whole cell vaccine approach that the cells used in a vaccine do not have to be identical to the tumor cells against which the vaccine is directed. For instance with pancreatic cancer, the initial approach in humans was to create a vaccine using autologous cells from each patient's tumor, but then it became apparent that a non-specific allogenic cell line could be used with similar success to the autogenous cells, and with much greater convenience (Jaffee, Hruban et al. 2001). There are at least two well known human esophageal adenocarcinoma cell lines, namely BIC and SEG. These cell lines would be candidates for use in a future human trial.

Potential targets for the esophageal cancer vaccine would be patients with an elevated risks of developing esophageal cancer. This could include patients with known risk factors such as gastro-esophageal reflux disease. The vaccination schedule and lack of toxicity of the vaccine provides physicians the opportunity to vaccinate individuals prior to any clinical signs of Barrett's. The use of this vaccine has the potential to reduce the reliance on therapies that have many unintended adverse effects.

In summary, this project was the first to develop a whole-cell GM-CSF vaccine to prevent esophageal cancer in animals. We were able to generate a stable esophageal cancer cell line. Although a GM-CSF secreting cancer vaccine derived from these cells was unsuccessful in preventing the development of Barrett's esophagus, the vaccine successfully inhibited carcinogenesis in a surgical rat reflux model. Our vaccine may represent a safe and potent option to prevent the development of esophageal cancer.

### REFERENCES

The disclosure of each reference cited is expressly incorporated herein.

### REFERENCES

1. Landis S H, Murray T, Bolden S, Wingo P A. Cancer statistics, 1998. CA Cancer J Clin 1998; 48:6-29.
2. Devesa S S, Blot W J, Fraumeni J F, Jr. Changing patterns in the incidence of esophageal and gastric carcinoma in the United States. Cancer 1998; 83:2049-53.
3. Landis S H, Murray T, Bolden S, Wingo P A. Cancer statistics, 1999. CA Cancer J Clin 1999; 49:8-31, 1.
4. Gibson M K, Forastiere A A. Combined-modality therapy for esophageal cancer: are we making progress? Cancer J 2003; 9:238-40.
5. Wild C P, Hardie L J. Reflux, Barrett's oesophagus and adenocarcinoma: burning questions. Nat Rev Cancer 2003; 3:676-84.
6. Byrnes C K, Bahadursingh A, Akhter N, Parinandi N L, Natarajan V, Montgomery E, Tihan T, Duncan M D, Nass P H, Harnon J W. Duodenal reflux produces hyperproliferative epithelial esophagitis—a possible precursor to esophageal adenocarcinoma in the rat. J Gastrointest Surg 2003; 7:172-80.
7. Bonde P, Sui G, Dhara S, Wang J, Broor A, Kim I F, Wiley J E, Marti G, Duncan M, Jaffee E, Montgomery E, Maitra A, Harmon J W. Cytogentic characterization and gene expression profilling in the rat reflux induced esophageal tumor model. J Thorac Cardiovasc Surg 2007; 133:763-9.
8. Buttar N S, Wang K K, Leontovich O, Westcott J Y, Pacifico R J, Anderson M A, Krishnadath K K, Lutzke L S, Burgart L U. Chemoprevention of esophageal adenocarcinoina by COX-2 inhibitors in an animal model of Barrett's esophagus. Gastroenterology 2002; 122:1101-12.
9. Miwa K, Sahara H, Segawa M, Kinami S, Sato T, Miyazaki I, Hattori T. Reflux of duodenal or gastro-duodenal contents induces esophageal carcinoma in rats. Int J Cancer 1996; 67:269-74.
10. Su Y, Chen X, Klein M, Fang M, Wang S, Yang C S, Goyal R K. Phenotype of columnar-lined esophagus in rats with esophagogastroduodenal anastomosis: similarity to human Barrett's esophagus. Lab Invest 2004; 84:753-65.
11. Sui G, Bonde P, Dhara S, Broor A, Wang J, Marti G, Feldmann G, Duncan M, Montgomery E, Maitra A, Harmon J W. Epidermal growth factor receptor and hedgehog signaling pathways are active in esophageal cancer cells from rat reflux model. J Surg Res 2006; 134:1-9.
12. Dranoff G, Jaffee E, Lazenby A, Golumbek P, Levitsky H, Brose K, Jackson V, Hamada H, Pardoll D, Mulligan R C. Vaccination with irradiated tumor cells engineered to secrete murine granulocyte-macrophage colony-stimulating factor stimulates potent, specific, and long-lasting anti-tumor immunity. Proc Natl Acad Sci USA 1993; 90:3539-43.
13. Jaffee E M, Hruban R H, Biedrzycki B, Laheru D, Schepers K, Sauter P R, Goemann M, Coleman J, Grochow L, Donehower R C, Lillemoe K D, O'Reilly S, Abrams R A, Pardoll D M, Cameron J L, Yeo C J. Novel allogeneic granulocyte-macrophage colony-stimulating factor-secreting tumor vaccine for pancreatic cancer: a phase I trial of safety and immune activation. J Clin Oncol 2001; 19:145-56.
14. Emens L A, Armstrong D, Biedrzycki B, Davidson N, Davis-Sproul J, Fetting J, Jaffee E, Onners B, Piantadosi S, Reilly R T, Stearns V, Tartakovsky I, Visvanathan K, Wolff A. A phase I vaccine safety and chemotherapy dose-finding trial of an allogeneic GM-CSF-secreting breast cancer vaccine given in a specifically timed sequence with immunomodulatory doses of cyclophosphamide and doxorubicin. Hum Gene Ther 2004; 15:313-37.
15. Simons J W, Jaffee E M, Weber C E, Levitsky H I, Nelson W G, Carducci M A, Lazenby A J, Cohen L K, Finn C C, Clift S M, Hauda K M, Beck L A, Leiferman K M, Owens A H, Jr., Piantadosi S, Dranoff G, Mulligan R C, Pardoll D M, Marshall F F. Bioactivity of autologous irradiated renal cell carcinoma vaccines generated by ex vivo granulocyte-macrophage colony-stimulating factor gene transfer. Cancer Res 1997; 57:1537-46.
16. Simons J W, Mikhak B, Chang J F, DeMarzo A M, Carducci M A, Lim M, Weber C E, Baccala A A, Goemann M A, Clift S M, Ando D G, Levitsky H I, Cohen L K, Sanda M G, Mulligan R C, Partin A W, Carter H B, Piantadosi S, Marshall F F, Nelson W G. Induction of immunity to prostate cancer antigens: results of a clinical trial of vaccination with irradiated autologous prostate tumor cells engineered to secrete granulocyte-macrophage colony-stimulating factor using ex vivo gene transfer. Cancer Res 1999; 59:5160-8.
17. Soiffer R, Lynch T, Mihm M, Jung K, Rhuda C, Schmollinger J C, Hodi F S, Liebster L, Lam P, Mentzer S, Singer S, Tanabe K K, Cosimi A B, Duda R, Sober A, Bhan A, Daley J, Neuberg D, Parry G, Rokovich J, Richards L, Drayer J, Berns A, Clift S, Cohen L K, Mulligan R C, Dranoff G. Vaccination with irradiated autologous melanoma cells engineered to secrete human granulocyte-macrophage colony-stimulating factor generates potent antitumor immunity in patients with metastatic melanoma. Proc Natl Acad Sci USA 1998; 95:13141-6.
18. Reed M F, Tolis G, Jr., Edil B H, Allan J S, Donahue D M, Gaissert H A, Moncure A C, Wain J C, Wright C D, Mathisen D J. Surgical treatment of esophageal high-grade dysplasia. Ann Thorac Surg 2005; 79:1110-5; discussion 1110-5.
19. Davis E A, Heitmiller R F. Esophagectomy for benign disease: trends in surgical results and management. Ann Thorac Surg 1996; 62:369-72.
20. Johnston M H. Technology insight: ablative techniques for Barrett's esophagus—current and emerging trends. Nat Clin Pract Gastroenterol Hepatol 2005; 2:323-30.
21. Levrat M, Lambert R, Kirshbaum G. Esophagitis produced by reflux of duodenal contents in rats. Am J Dig Dis 1962; 7:564-73.
22. Attwood S E, Smyrk T C, DeMeester T R, Mirvish S S, Stein H J, Hinder R A. Duodenoesophageal reflux and the development of esophageal adenocarcinoma in rats. Surgery 1992; 111:503-10.
23. Chen X, Yang G, Ding W Y, Bondoc F, Curtis S K, Yang C S. An esophagogastroduodenal anastomosis model for esophageal adenocarcinogenesis in rats and enhancement by iron overload. Carcinogenesis 1999; 20:1801-8.
24. Pera M, Brito M J, Poulsom R, Riera E, Grande L, Hanby A, Wright N A. Duodenal-content reflux esophagitis induces the development of glandular metaplasia and adenosquamous carcinoma in rats. Carcinogenesis 2000; 21:1587-91.
25. Miyasbita T, Ohta T, Fujimura T, Ninomiya I, Fushida S, Hattori T, Miwa K. Duodenal juice stimulates oesophageal stem cells to induce Barrett's oesophagus and oesophageal adenocarcinoma in rats. Oncol Rep 2006; 15:1469-75.
26. Nishijima K, Miwa K, Miyashita T, Kinami S, Ninomiya I, Fushida S, Fujimura T, Hattori T. Impact of the biliary diversion procedure on carcinogenesis in Barrett's esophagus surgically induced by duodenoesophageal reflux in rats. Ann Surg 2004; 240:57-67.
27. Giuli R, Scarpignato C. The Duodenogastroesophageal reflux: From the duodenum to the trachea. 125 questions-125 answers. John Libbery Eurotext, 2006
28. Thomas M C, Greten T F, Pardoll D M, Jaffee E M. Enhanced tumor protection by granulocyte-macrophage colony-stimulating factor expression at the site of an allogeneic vaccine. Hum Gene Ther 1998; 9:835-43.
29. Miyashita T, Furhawn Shah J W, Todd Armstrong, Pramod Bonde, Michael K Gibson, Kiyoshi Yoshimura, Xianjie Zhang, Lixin Liu, Guy Marti, Elizabeth A. Montgomery, Mark Duncan, Elizabeth M. Jaffee, John W Harmon. Granulocyte Macrophage Colony Stimulating Factor (GM-CSF) Transfected Whole Cell Vaccine Impedes the Development of Reflux Induced Esophageal Cancer in a Surgical Rat Model. J Gastrointest Surg 2007.

1. A composition comprising mammalian esophageal cancer cells which express MHC I molecules and which express GM-CSF from an exogenous expression construct.

2. The composition of claim 1 wherein the cells are propagation-defective.

3. The composition of claim 1 wherein the expression construct is a plasmid.

4. The composition of claim 1 wherein the GM-CSF is expressed from a viral promoter.

5. The composition of claim 1 wherein the cancer cells are human.

6. The composition of claim 1 wherein the cancer cells are rat.

7. The composition of claim 1 wherein the GM-CSF is human GM-CSF.

8. The composition of claim 1 wherein the GM-CSF is rodent GM-CSF.

9. The composition of claim 1 wherein the cancer cells are irradiated.

10. The composition of claim 1 wherein the propagation-defective cells can propagate in neither in vitro nor in vivo conditions.

11. The composition of claim 1 wherein the cancer cells are derived from a primary tumor with adenosquamous histology.

12. The composition of claim 1 wherein the cancer cells are derived from a primary tumor with adenocarcinoma histology.

13. The composition of claim 1 wherein the cancer cells are derived from a primary tumor with squamous cell histology.

14. A method of treating a mammal with esophageal cancer, with Barrett's metaplasia, or prone to develop Barrett's metaplasia, comprising:
administering the composition of claim 1 to the mammal whereby risk that the mammal develops esophageal cancer, esophageal cancer recurrence, or esophageal cancer metastases is reduced.

15. The method of claim 14 wherein 10⁶ to 5×10¹⁰ propagation-defective mammalian esophageal cancer cells are administered.

16. The method of claim 14 wherein 10⁸ to 10⁹ propagation-defective mammalian esophageal cancer cells are administered.

17. The method of claim 14 wherein 10⁹ to 10¹⁰ propagation-defective mammalian esophageal cancer cells are administered.

18. The method of claim 14 wherein the mammal is prone to develop Barrett's metaplasia.

19. The method of claim 14 wherein the mammal is prone to develop Barrett's metaplasia by virtue of having chronic gastroesophageal reflux disease.

20. The method of claim 14 wherein the mammal has Barrett's metaplasia.

21. The method of claim 14 wherein the mammal has had resection of an esophageal tumor.

22. The method of claim 14 wherein the mammal has an esophageal tumor.

23. The method of claim 14 wherein the mammal and the cancer cells are allogeneic.

24. The method of claim 14 wherein the mammal and the cancer cells are xenogeneic.

25. The method of claim 14 wherein the step of administering is repeated.
